(19) United States
(12) Patent Application Publication
(10) Pub. No.: US 2014/0243789 A1
(43) Pub. Date: Aug. 28, 2014

(54) Subcutaneous Dialysis Catheter with Ultrasound Agitation

(71) Applicants: Neerav Mehta, Swarthmore, PA (US); Steven Short, Hartfield, VA (US); David Smoger, Villanova, PA (US)

(72) Inventors: Neerav Mehta, Swarthmore, PA (US); Steven Short, Hartfield, VA (US); David Smoger, Villanova, PA (US)

(21) Appl. No.: 14/191,397

(22) Filed: Feb. 26, 2014

Related U.S. Application Data
(60) Provisional application No. 61/769,963, filed on Feb. 27, 2013.

(51) Int. Cl. A61M 1/36

(52) U.S. Cl. CPC A61M 1/3672
USPC 604/508; 604/175

(57) ABSTRACT

A subcutaneous, venous catheter is provided in conjunction with a method of installation in hemodialysis treatments. The catheter has an implantable hub attached to a first end of the primary lumen. An anchoring back plate is pivottably secured to the catheter hub and surgically anchored to underlying musculature. Once the device is implanted, the hub can be arcuately translated underneath the skin by applying gentle pressure to either side of the hub. To reduce fluid stagnation within and around the lumen, a series of piezo-electric elements are integrated therein. A vibration processor is electrically connected to the piezo-electric elements such that the initiation of electrical current by the vibration processor results in contraction of the piezo-electric elements. Expansion and contraction of these elements propagates low-energy acoustic waves through the lumen, agitating liquid contained therein and improving flow of same through the catheter lumen.

### CROSS REFERENCE TO RELATED APPLICATION

This application claims the benefit of U.S. Provisional Application No. 61/769,963 filed on Feb. 26, 2013, entitled “Subcutaneous Dialysis Catheter with Ultrasound Agitation.” The patent application identified above is incorporated here by reference in its entirety to provide continuity of disclosure.

### BACKGROUND OF THE INVENTION

1. Field of the Invention

The present invention relates to the field of implantable therapeutic agent delivery devices. More specifically, the invention relates to subcutaneous, venous catheters for localized therapeutic agent delivery and blood filtration. The present invention is a subcutaneous, venous catheter with a repositionable catheter hub, and mechanical flow actuation and excitation inducing elements, as well as an associated method of installation.

Blood filtration, known as hemodialysis, is used to treat patients experiencing renal malfunction or failure. Hemodialysis treatments remove excess fluids, salts, and wastes such as urea and creatinine from a patient's blood supply by pumping it through specialized filters. Treatment regiments are generally prescribed for a daily, nightly, or three times a week basis, thereby avoiding the potentially fatal over-accumulation of wastes within the blood stream. The duration and frequency of filtration depends on a patient's individual body chemistry, renal function, and the type of hemodialysis employed. Strict adherence to predetermined treatment time schedules requires ready access to a patient's blood supply and a minimal amount of trauma to patient vasculature during each hemodialysis session.

Vascular access needed for hemodialysis treatments is established through one of three methods. Intravenous catheters, a lumen inserted into a blood vessel; arteriovenous (AV) fistulae, the merging of an artery and a vein to form an organic lumen for blood filtration; and artificial grafts, fistulae formed from synthetic or animal vessels, are the primary methods of vascular access used in hemodialysis treatments. The preferred access method is creation of AV fistulae, because of the low risk of complication they present. But, not all patient anatomies are conducive to fistulae creation, and even successfully matured AV fistulae fail over time. Grafts may be attempted on patients whose vascular architecture is insufficient for creation of natural fistulae, or may be used to supplement and replace failed AV fistulae. Over time, grafts too will fail or narrow to the point where blood flow through the tunnel is insufficient for hemodialysis treatments. Catheters, as the method of last resort, often become a necessity for those undergoing long-term hemodialysis.

Central venous catheters (CVC) are bi-lumenal or mono-lumenal vascular access lines. They are frequently used for blood filtration and localized delivery of therapeutic agents, and for diagnostic testing of vascular blood. When deployed, the primary lumen is inserted into the internal jugular, subclavian, or femoral vein of a patient, where it remains throughout treatments. External lines may be connected to the primary lumen during hemodialysis sessions, thereby connecting the catheter to the filtration circuit and facilitating the flow of blood therethrough.

Venous catheters utilized in the hemodialysis process are commonly classified as tunneled, non-tunneled, or implanted. Non-tunneled catheters are only optimal for single use dialysis sessions, because the primary lumen and associated connections are extracorporeal, and are easily dislodged from the access site if snagged on objects in the surrounding environment. Conversely, tunneled catheters are surgically installed in a subcutaneous position with exit ports protruding from the skin, thereby reducing potential for trauma resulting from violent lumen dislodgment. But, the external access ports of tunneled catheters are prone to failure secondary to infection; lumenal blood clotting (thrombosis) and fibrin sheath formation and as a result are suboptimal long-term access solutions. As a result of the long-term use risks, tunneled catheters are ideal for use in patients experiencing acute renal failure from which they are likely to recover all or part of normal renal function, or those patients waiting on an AV fistulae or graft to fully mature.

Internal port, or implanted catheters have access ports implanted under the skin. Implant ports are in fluid communication with an attached reservoir, facilitating temporary storage of therapeutic agents, and enabling timed agent release options. For these reasons, implant catheters are also used for some chemotherapy treatments and other long-term therapeutic agent delivery regiments, in addition to hemodialysis. During treatments, catheter ports are accessed via needles inserted through the skin. Injection sites are easily sterilized and do not remain open between treatments, thereby reducing risk of bacterial infection. Though less susceptible to infection than tunneled catheters, completely subcutaneous catheters are equally susceptible to lumenal thrombosis and fibrin sheath formation. Saline solution is flushed through the primary lumen between hemodialysis treatments to help prevent platelet and fibrin accumulation, but clot formation remains a serious risk and may lead to vessel occlusion if complicated by venous stenosis.

The amalgamation of platelets and fibrin into blood clots known as thrombi naturally occurs upon trauma to the vasculature. Thrombus formation is achieved through polymerisation of fibrogen and thrombin, which coalesce into a fibrin mesh. This mesh forms over damaged portions of blood vessels, protecting the vasculature walls and thereby assisting in healing of damaged endothelial cells. Thrombus creation is not limited to instances of vasculature trauma, and can also occur secondary to hypercoagulatory conditions (thrombophilia), artificially reduced blood flow, organ failure or vascular disease. Thus, the creation of thrombi is a natural response to internal injury within the body.

Despite the therapeutic nature of thrombi, the formation of oversized or overabundant blood clots poses serious health risks to patients. Blood flow reduction (ischemia), stasis, or stagnation reduces effectiveness of oxygenation, thereby increasing stress on a patient's cardiovascular system, which must work harder to push blood through the affected area. Without adequate oxygen, surrounding tissue will become necrotic (infarction), potentially leading to organ failure and other serious health risks. Formation of a thrombus may reduce blood flow by obstructing all or part of a blood vessel (occlusion) or may exacerbate existing blood flow stagnation issues. Vasculature compression caused by cancerous growths, and tumors may lead to reduced blood flow, while the release of procoagulant substances by cancer cells can increase the likelihood of local thrombus formation. Arterial fibrillation can bring on sluggish blood flow within the left atrium, increasing the risk of thrombi formation within the heart. Hypercoagulatory conditions can result from autoimmune disease; genetic deficiency, chemotherapy and radiation treatments also present heightened risk of blood clot formation because the bloodstream is predisposed to clumping. The presence of a catheter lumen within an area affected by thrombosis further increases the risk of serious complication because partial occlusion of the vessel is already achieved during lumen insertion.

Blood vessel occlusion resulting from thromboembolytic blockages poses a particularly serious threat to a patient's health. Emboli are vascular blockages, which can be caused by thrombi that detach from blood vessel walls, generally during normal thrombi recanlization, and travel through the blood stream (thromboembolism). These free-floating masses of fibrin can lodge in blood vessels far from their origination point as well as local vasculature. If not treated quickly, embolytic occlusion can lead to severe ischemia and eventual tissue necrosis in the affected area (infarction). Stroke and myocardial infarction (heart attack) are few of the life-threatening conditions that result from Incidents of thromboembolytic ischemia in the brain and heart. Retinal (eye) and renal (kidney) embolism can produce painful, long-term health problems that reduce quality of life for affected individuals. The deleterious effects of thromboembolism can be experienced throughout the cardiovascular system, and are not limited to the organs discussed above. Patients suffering from ischemia or infarction can experience symptoms ranging from pain, loss of function, blindness, organ failure, to death.

Thrombolytic drugs (clot dissolving are often used by surgical patients and implant recipients to break up blood clots, and prevent the formation of new clots. These treatments are not without risk of complication. Thrombolytic medications can cause excessive bleeding both internal and external upon the infliction of even minor trauma. Patients receiving venous catheters also take anti-coagulant medications for a period after implantation to reduce the risk of thrombi formation. Like thrombolytics, these medicines can cause excessive, potentially serious bleeding.

An additional complication of implanted catheters is the pain associated with regular needle injections into a subcutaneous port. During each hemodialysis session or therapeutic agent delivery, needles are inserted through the same portion of the patient's skin. Repeated injection into the same site can lead to bruising, soreness, and swelling, making implanted catheters unsuitable for patients needing frequent dialysis treatments.

An implanted venous catheter that provides lumen agitation and repositionable injection ports is needed to reduce the risk of thrombosis and fibrin sheath formation, as well as minimizing the pain and discomfort associated with treatment sessions. By reducing the health risks and pain secondary to implant catheter usage, the needed device enables frequent use over longer periods.

2. Description of the Prior Art

The present invention is a subcutaneous venous catheter having a repositionable hub and piezo-electric agitators, to reduce pain associated with dialysis sessions and the overall risk of thrombus or fibrin sheath formation during the device's lifespan. A method of utilizing the catheter in hemodialysis is provided to guide practitioners in best practices for long-term use of the device. The catheter has a primary lumen secured at a first end to a catheter hub comprising a fluid reservoir and attached injection port. The hub is pivotably attached to an anchoring back plate to permit subcutaneous translation of the reservoir and injection port with respect to the skin. To anchor the device in a desired position, the back plate is surgically attached to underlying musculature during implantation of the device. Once implanted, the injection port is repositioned underneath a patient's skin via the application of gentle pressure on either side of the catheter hub.

A series of piezo-electric elements are integrated throughout the primary lumen length. These piezo-electric elements are electrically connected to a vibration processor that initiates the flow of electrical current through the primary lumen. Electrical flow through the piezo-elements results in ultrasonic vibrations. These vibrations promote fluid translation throughout the primary lumen, and reduce stagnation of blood in the surrounding venal canal. Frequency and duration of lumen agitation is controlled by the vibration processor and may be controlled by an attending physician. The prior art does not teach a catheter device having a repositionable implanted access port and a plurality of integrated piezo-electric elements. The following list of references is a list of the prior art considered relevant to the present disclosure.

Lumens containing piezo-electric elements have been used in urinary catheters to reduce the accumulation of bacteria laden bio-film. Because urinary catheters are inherently non-implantable, they are exposed to bacteria, which can lead to sepsis if allowed to accumulate and proliferate. Use of piezo-electric elements to gently vibrate the lumen, thereby agitating fluid in the urethra and creating an environment hostile to bacteria growth. Examples of these urinary catheters can be found in Zumeris, U.S. Pat. No. 7,393,501 and Zumeris, U.S. Pat. No. 7,829,029. These catheters are not subcutaneous and do not include implanted access ports nor do they disclose pivotably re-positionable ports anchored to living tissue.

Catheters containing piezo-electric elements have also been used in the diagnostic testing of intravascular lesions. Sanatjian, U.S. Pat. No. 7,291,110, discloses an apparatus and associated method of utilizing ultrasonic vibrations along a catheter lumen to map vascular lesions. The device is an expandable balloon type catheter having a lumen with a plurality of piezo elements integrated into the lumen surface. This catheter is inserted into a vessel along a guiding wire, to bring the expandable lumen surface into contact with a vessel-occluding lesion. A first portion of the integrated piezo elements is initialized, transmitting acoustic waves into the lesion. A second portion measures returned wave patterns. Reflection and refraction of the sound waves is dependent upon the material composition of the lesion and the disposition of transmitting piezo elements. Received wave information is processed externally to assess the topography and composition of the lesion. The Sanatijian device is not a subcutaneous catheter, and does not include access ports, pivotable reservoirs or any subcutaneous anchoring means for the catheter.

Intravenous catheters containing piezo-electric elements have been disclosed for the use of therapeutic agent delivery in Brisken, U.S. Pat. No. 5,735,811 and Homsma, U.S. Pat. No. 5,928,186. Specifically, Brisken teaches an intravenous catheter device and associated method of using mechanical vibrations to ablate and dissolve vascular occlusion caused by stenotic lesions, accumulated arteriovenous plaque, and thrombi. The mechanical vibrations of the Brisken catheter are generated using piezo-electric elements and may be used without employing therapeutic agents, or in combination with thrombolytic agents. To this end, the configuration of the piezo-electric elements of Brisken is specifically designed to create outwardly radial vibrations that penetrate occluding material. Conversely, the present invention includes piezo-electric elements configured to induce longitudinal wave propagation, primarily directed inwards, to agitate therapeutic agents contained within the lumen and resultantly improve fluid flow therethrough. Thus, the device of Brisken is unsuitable for the purpose of aiding in therapeutic agent delivery during hemodialysis, because it does not operate to accelerate the flow of fluids through the lumen.

The Homsma device is another intravenous catheter configured to ablate and dissolve vascular occlusions. Unlike the Brisken device, Homsma teaches the propagation of longitudinal waves throughout a catheter lumen via a plurality of piezo-electric element disposed at a first or second end of the lumen. A conical mirror attachment disposed at an end of the catheter lumen distal from the high-frequency generator, deflects ultrasonic waves outward into a target area. Homsma does not disclose a series of piezo-electric elements integrated into and disposed along the length of a catheter lumen.

Both the Brisken device and the Homsma device are unsuitable for use as an implanted intravenous catheter. The energy needed to create ultrasonic waves that propagate along the length and exit the distal end with sufficient strength to affect dissolution of occlusion material is greater than that needed by the present invention, which does not destroy surrounding material; rather it agitates fluid within the lumen to reduce stagnation. As such, the present invention can offer improved battery life and is suitable for implant grade catheters, which must operate on an onboard battery for a sustained duration of time. The Brisken and Homsma devices may be useful in thrombolectomy procedures, but are not viable as long-term hemodialysis treatment options.

Further, the neither Homsma nor Brisken teaches an implanted catheter hub that is repositionable with respect to an anchored back plate. Resultantly, these devices are used in surgical procedures and are not intended or adapted for use as a semi-permanent medical apparatus. The present invention solves these problems by providing an implantable injection ports and an onboard battery.

These prior art devices have several known drawbacks. They do not disclose an intravenous catheter having implantable injection ports, a repositionable hub secured to an anchoring back plate, or fluid agitation via piezo-electric elements. The present invention incorporates these elements into a device and method of use, in order to facilitate long-term hemodialysis treatments. It substantially diverges in design elements from the prior art and consequently it is clear that there is a need in the art for an improvement to existing therapeutic agent delivery devices. In this regard the instant invention substantially fulfills these needs.

### SUMMARY OF THE INVENTION

In view of the foregoing disadvantages inherent in the known types of therapeutic agent delivery devices now present in the prior art, the present invention provides a new repositionable reservoir and uniquely configured piezo-electric elements, wherein the same can be utilized for providing convenience for a patient undergoing long-term hemodialysis treatments.

The present invention is a venous catheter that is implanted subcutaneously, thereby limiting exposure to external bacteria and subsequently reducing the risk of infection. Access to the catheter is achieved by puncturing the skin lying directly over the injection port of the catheter hub, which is pivotably secured at least one point to an anchoring back plate, thereby facilitating translation of the catheter hub in an arc along at least one axis. Doctors and caregivers can manipulate the catheter hub underneath a patient's skin to shift the location of the injection ports and reduce the frequency of injecting into the same area of skin. The catheter is exposed to the outside environment only during hemodialysis treatments or agent delivery, and only through the injection sites, which can be sterilized before and after treatments.

Additionally, the catheter has piezo-electric rings embedded within the walls of the primary lumen to improve the flow of therapeutic fluids therethrough. This is accomplished by initiating electrical pulses that run along the length of the catheter lumen causing piezo-element expansion/contraction and stimulating fluid flow through the primary lumens. The agitation creates a motion that promotes intravascular fluid flow as well as intralumenal fluid flow. In this way, the invention helps reduce clotting along the catheter course.

The catheter device is surgically installed under a patient's skin. An operating surgeon first makes an incision into the patient's skin and into a target vascular pathway. One or more guide wires are fed into the vascular pathway until a desired depth of insertion is achieved. Next second ends of the lumens are fed along the guide wire and into the vascular pathway. The first ends of the lumens, having connection cuffs for removably securing the lumens to the hub are trapped beneath the lumen brackets of the anchoring back plate. The plate is attached to underlying fascia via suturing a plurality of suture wings to exposed tissue. Next the first ends of the lumens are connected to the hub, thereby securing the lumens to the hub's fluid reservoirs and electrically connecting the lumens to the power source. Finally, the hub is removably secured to the anchoring back plate by inserting the male extension of the hub into the upstanding collar of the anchoring back plate and pressing the two together. The hub will rotate about the male extension trapped within the upstanding collar, enabling repositioning of the injection ports. Finally the lumens are fed all the way into the vascular pathway, and the wounds are closed.

During a hemodialysis session the each of the injection ports is accessed to create a blood withdrawal and blood delivery line. Blood is withdrawn from the body, filtered and returned to the body. In between treatments, the hub's internal reservoirs are filled with saline solution to facilitate flushing the lines. In the primary embodiment, removal of the injection needle from the injection ports initiates electrical flow to the piezoelectric elements integrated along the length of the lumens. The expansion and contraction of the transducer array piezo-elements induces wave propagation throughout the liquid rerunning through the lumens. Encouraging fluid movement through the lumens in between treatments reduces bacterial growth and the likelihood of thrombi formation.

It is therefore an object of the present invention to provide a new and improved therapeutic agent delivery device that has all of the advantages of the prior art and none of the disadvantages.

It is therefore an object of the present invention to provide an intravenous catheter that addresses the risks of both blood clot formation and bacterial infection, thereby rendering the device suitable as a long-term hemodialysis treatment option.

Another object of the present invention is to provide an implantable venous catheter with a subcutaneous catheter hub that is repositionable, and thus reduces the frequency of use of any injection site. By occasionally repositioning the injection sites with respect to a patient's skin, the attending physician can reduce the pain and discomfort associated with regular hemodialysis treatment sessions.

Yet another object of the present invention is to provide a venous catheter with a subcutaneous hub, to reduce catheter exposure to environmental contaminants.

Still another object of the present invention, is to provide an implanted, subcutaneous catheter adapted to agitate fluids within the lumen in order to reduce fluid stagnation and improve delivery of therapeutic agents.

A further object of the present invention is to provide a venous catheter adapted to agitate intravascular fluids and resultantly reduce the likelihood of thrombi formation.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a side view of the partially assembled hemodialysis catheter. The hub and lines of the venous catheter are connected and ready for use.

FIG. 2 shows an overhead view of the assembled hemodialysis catheter with injection ports and associated conduit tunnels visible.

FIG. 3 shows a prospective view of an exemplary lumen of the line assembly. The lumen has an integrated transducer array with a plurality of piezo-electric elements distributed throughout the lumen length.

FIG. 4 is a cross-section view of an exemplary configuration of the line assembly of the present invention.

FIG. 5 shows an overhead view of the anchoring back plate of the present invention.

FIG. 6 shows a perspective view of the anchoring back plate. The upstanding collar extends outward from the upper surface if the back plate.

FIG. 7 shows a cross-section view of the catheter hub.

FIG. 8 shows a perspective view of the catheter hub being attached to the anchoring back plate, which is sutured into position. The line assembly is in the process of connecting to the connection ports of the catheter hub.

FIG. 9 shows a flow chart of the method of installing the subcutaneous hemodialysis catheter device in a patient.

### DETAILED DESCRIPTION OF THE INVENTION

Reference is made herein to the attached drawings. Like reference numerals are used throughout the drawings to depict like or similar elements of the subcutaneous hemodialysis catheter device. For the purposes of presenting a brief and clear description of the present invention, the preferred embodiment will be discussed as used for hemodialysis treatments. The figures are intended for representative purposes only and should not be considered to be limiting in any respect.

The present invention provides a hemodialysis catheter device and associated method of installation. The catheter generates low-energy acoustic vibrations that reduce intralumenal fluid stagnation, prevent microbial biofilm formation, and dissolve incidences of vascular occlusion. A transducer array of piezoelectric elements is integrated throughout each of the catheter lumens generate low-energy acoustic waves upon initiation of electrical current flow. Multiple types of piezoelectric elements are utilized in order to provide varied acoustic wave characteristics capable of accomplishing the aforementioned objectives.

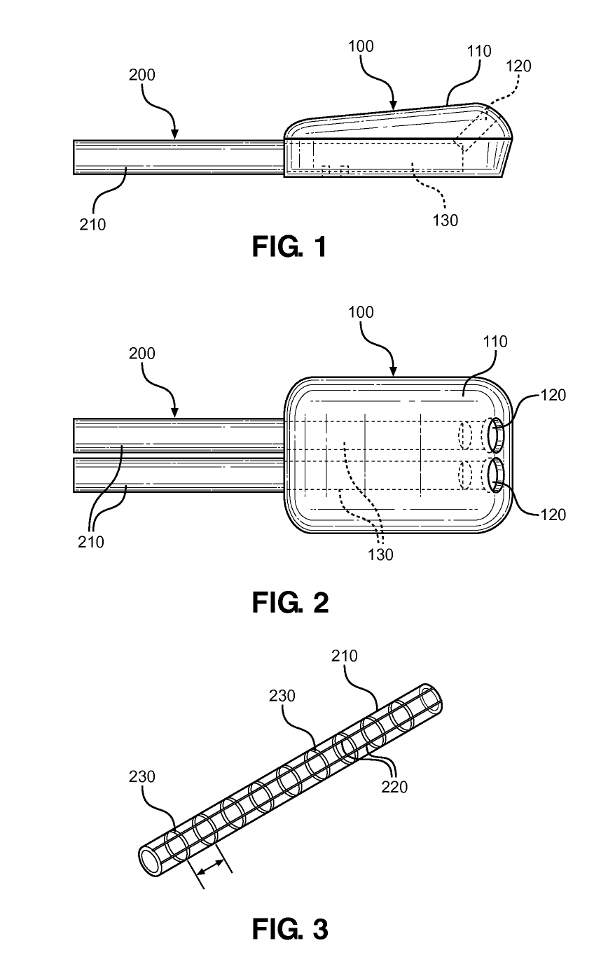

Referring now to FIG. 1, there is shown a side view of the partially assembled catheter device. A central venous catheter hub 100 is connected to a line assembly 200 having a bi-lumenal configuration. A pair of injection ports 110 extends inward from an upper surface of the hub body 110 upper end, which lies distal to the line assembly. Lying horizontal and connected to the end of the injection ports Isa pair of conduit tunnels 130. These conduit tunnels are separate and distinct from each other, rendering the catheter hub bi-cameral. Opposing ends of the conduit tunnels terminate in connection ports that facilitate removable engagement of the line assembly to the hub body. Thus the conduit tunnels place the line assembly lumens in fluid communication with the conduit tunnels and injection ports. Geometry and volume of the conduit tunnels within the hub body may vary according to the desired volume of liquid retention. Optionally, The uppermost surface of the hub body may taper from the injection ports down to the end proximal to the line assembly to reduce the hub's volume. This shape also aids physicians in locating the injection ports after device implantation, because the portion of the hub body containing the injection ports will protrude slightly from below a patient's skin.

Each of the injection ports is positioned at an angle of approximately thirty degrees from the horizontal. Angling the injection ports in this manner improves liquid delivery by providing initial forward momentum to delivered fluid. In contrast, injection ports that are orthogonally positioned with respect to the horizontal deliver liquids with momentum normal to the flow of liquid within the associated conduit tunnel. Thus, the angled injection ports reduce turbulent flow within the fluid stream and increase laminar flow in same.

Catheter lumens of the line assembly lie in a parallel configuration as shown in FIG. 2. Similarly, the conduit tunnels 130 within the hub body 110 run in parallel between the injection ports 120 and the connection ports, where the conduit tunnels connect to the lumens 210 of the line assembly 200. Connection between the catheter hub 100 and the lumens may be accomplished through any means known in the art of intravenous therapeutic agent delivery devices. To enable easy installation of the device, it is preferred that the lumens connect and disconnect from the connection ports with minimal physical manipulation. By way of example, the connection port may have an interior flange, through which a collapsible flange on the first end of a lumen is inserted. After passing through the interior flange of the connection port, the collapsible flange of the lumen expands, temporarily engaging connection between the lumen and the hub body. Preferably, the lumen may be removed from the connection port via depression of an exterior portion of the lumen first end, which results in sufficient constricting of the collapsible flange to permit it to slip backward through the connection port interior flange. Alternative connection means such as female screw threading within the connection port and male screw threading disposed on the first end of the lumen, or snap in configurations, may also be employed.

All embodiments of catheter hub connection ports provide a pathway for electrical current to flow from a power source (not shown) through the connection ports to the line assembly. The first end of each lumen may have a small metal ring extending wholly or partially through the lumen walls, and exposed along the lumen outer surface. Multiple wires running throughout the length of each lumen are in electrical communication with the metal ring. When the lumen is properly fitted within a connection port, the exposed metal of the metal ring lies in connection with an exposed metal surface within the connection port. This exposed metal surface within the connection port is electrically connected via an integrated wire to an activator and power source. In this manner, electrical current flows from the battery source to the activator, through the connection port, and into the line assembly lumens. Initiation and termination of electrical current flow may be accomplished through injection port access, as is discussed in more detail below.

The line assembly 200 is depicted comprising two discrete lengths of tubing forming a parallel bi-lumenal structure. The lumens may be attached to each other with thin, flexible brackets, such as those made from thin plastics; or alternatively may be joined together with adhesive or bonding material. In alternative embodiments, the line assembly may consist of a single piece of tubing that is bisected into two distinct lumen pathways. Configuration of the transducer array is modified to integrate the piezoelectric elements into the primary tubing wall and the interior division.

Turning now to FIG. 3 one of the catheter lumens is shown with piezoelectric transducer array and associated wiring. A plurality of piezoelectric elements form a transducer array 230, which is integrated into the walls of each lumen 210. Two or more electrical wires extend throughout each lumen, contacting each piezoelectric element, thereby placing the elements of the transducer array in electrical communication. Desired resonance and wave propagation mode of the piezoelectric transducers may determine the shape and size of individual elements. In the preferred embodiment, acoustic wave propagation is directed longitudinally or radially. Longitudinal wave propagation provides fluid agitation and thus promotes intralumenal fluid flow along the lumen length. Radially directed wave propagation may be better suited to inhibiting biofilm formation and dissolving vascular occlusions. Piezoelectric elements within the illustrated transducer arrays are torus shaped, but spherical, conical, and rectangular elements as well as those having irregular geometries may also be employed. In all embodiments, piezoelectric elements and the wires should be insulated from the surrounding intravascular environment. Techniques for molding electrical elements into lumens, as well as dipping in and painting of elements with insular materials are known in the art and will be readily apparent to one of ordinary skill.

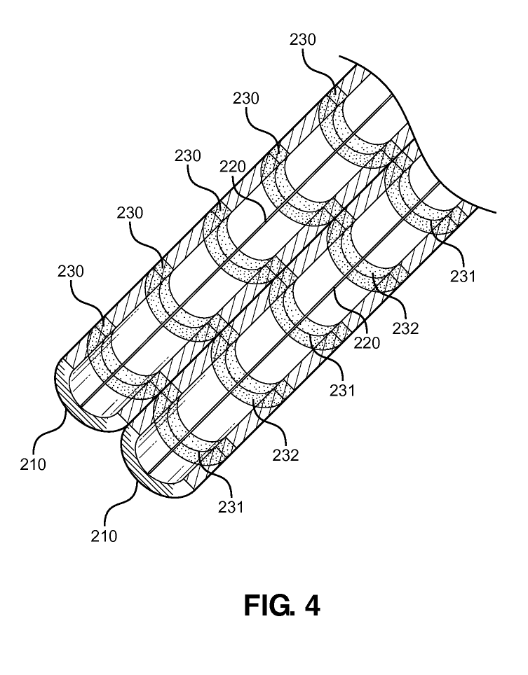

A cross-section of the two lumens of the line assembly is shown in detail in FIG. 4. A portion of an exemplary piezoelectric transducer array 230 is depicted, with individual piezoelectric elements 231, 232 disposed in a linear alignment along lumen 210 lengths. Materials having crystal structure known to exhibit piezoelectric behavior may be used in the construction of the transducer array elements. In some embodiments, two distinct piezoelectric elements are contained in the array.

A first set of piezoelectric crystals 231 is constructed of a material and shaped such that the elements resonate at a frequency of 300-700 kHz. Propagated waves having frequencies within this range, particularly those falling between 340-500 kHz have been shown to reduce the mass of vascular occlusions to which they are applied. Excitation of the first set of piezoelectric elements by the activator results in propagation of acoustic waves through the lumens and into the surrounding vascular environment. Application of waves having a frequency of 300-700 kHz over a prolonged period of several hours or more, can wholly or partially dissolve existing thrombi and inhibit new thrombi formation. Tissue surrounding the line assembly may have a dampening effect on wave propagation and thus the duration of time needed to affect thrombi mass reduction will be dependent upon occlusion characteristics and the frequency of acoustic wave applied.

A second set of piezoelectric crystals 232 is included in the piezoelectric transducer array 230. The piezoelectric crystals of the second set are constructed of and shaped to resonate at a frequency of 100-300 kHz. Acoustic wave applications within this range have been shown to inhibit bacterial adhesion to red blood cells and tissue. Prolonged excitation of the second set of piezoelectric crystals results in wave propagation along and around the lumen and can reduce biofilm formation within the lumen and along its exterior surface area.

It has been suggested that acoustic waves having energy higher than 0.35 mW/cm² may actually induce bacterial adhesion to red blood cells and tissue. As such, it is recommended that the first set of piezoelectric crystal elements resonate in the lower end of the 300-700 kHz range during regular operation. For the purposes of illustration, a frequency of 350 kHz may be used to reduce thrombi mass, without interfering with the bacterial adhesion inhibition of the second set of piezoelectric crystals.

Configuration of piezoelectric crystal elements 231, 232 within the transducer arrays may be adjusted to achieve optimal acoustic wave propagation throughout the lumens and the surrounding environment. In some embodiments, the elements of the transducer array may alternate between elements from the first set and elements from the second set. Other variations include the inclusion of only the first set of piezoelectric elements, only the second set of piezoelectric elements, or a transducer array in one lumen containing only piezoelectric elements from the first set, while the other lumen contains piezoelectric elements of the second set. Any other order of piezoelectric elements along the catheter lumens 210 is also contemplated. In all configurations, the transducer array elements should be positioned to enable wave propagation throughout liquid contained within the lumen. To this end, the transducer arrays of the parallel lumens should be configured to generate waves that are synchronized for parallel wave propagation or constructive interference. Destructive interference between the two transducer arrays should be avoided.

Wires 220 connect the piezoelectric transducer array elements to each other and to the metal ring disposed at the first end of the lumens. When the lumens are connected to the hub body, exposed metal surfaces within the connection ports contact the metal rings of the lumens, enabling the flow of electrical current therebetween. An activator and battery (not shown) or any other source of electrical current known to one of skill in the art, are electrically connected to the exposed metal surfaces within the connection ports. In this way, the transducer array elements receive electrical flow from the power source, as directed by the activator.

The activator is preferably an oscillating circuit, with voltage amplification. An astable multivibrator driver and the battery are electrically coupled to the acoustic wave generation elements via the electrical wires, metal rings, and exposed metal surfaces of the connection ports. Exact design of the electrical couplings may involve any configuration and material construction known to one of ordinary skill in the art. An exemplary acoustic wave generation controller is a printed circuit board with attached chips and timing mechanism. This controller is configured to operate a duty cycle of the acoustic wave generation. Although this configuration is preferred because it does not require the use of a microcontroller and thus improves battery life, alternate embodiments of the catheter device contemplate the use of microcontrollers in acoustic vibration generation management. The control panel, activator and battery are integrated into the hub body, or may be secured to the exterior of same and insulated from the surrounding environment via dipping, molding, painting, or any other insulating means known in the art.

Initiation of acoustic vibration may be affected via removal of a needle from one or both of the injection ports. The injection ports may contain pressure-sensitive plates that are in electrical communication with the activator. Alternatively, each injection port may contain electrical connections coupled to the activator such that needle insertion into the injection port completes a circuit and signals termination of transducer array agitation. In another alternative, acoustic wave generation is initiated and terminated via a depressible button disposed on the upper surface of the catheter hub. The button is electrically connected to the activator. A first button depression initiates electrical flow to the transducer arrays, and a second button push terminates electrical flow. The button is accessible via exertion of downward force on the area of skin lying over the implanted catheter hub and button.

During hemodialysis treatments, blood is withdrawn from the body through one lumen, passed through a filtration machine, and reincorporated into the body via the other lumen. Needles are inserted into the injection ports to enable blood transfer from the catheter device to the filtration machine. The speed at which fluid flows in and out of the catheter is largely determined by the filtration device's blood processing rate. Fluid flow within the catheter lumens should not be encouraged or impeded by acoustic wave propagation during treatments as this may interfere with the rate of fluid flow into and from the filtration machine. Thus, transducer array activation is re-initiated after a treatment session, upon removal of all needles from the injection ports or depression of a button.

The catheter device is powered by a battery or other power source. The battery may be 9.0V or higher and is rechargeable. Medical implant batteries have been successfully charged transcutaneously via current-pumped battery chargers (CPBC). As an example a 100 mAh battery can be transcutaneously charged within 137 minutes, with a charging efficiency of 85%. Tissue temperature during charging does not rise above a 2.1° C. change. Inductive charging is therefore considered the preferred method of battery charging as it presents minimal risk to the patient, and does not involve exposure of the hemodialysis catheter device to the outside environment. If inductive chargers are not available, or the battery is damaged, the hub body can be easily replaced without requiring major surgery. Because the catheter hub disconnects from the line assembly and the anchoring back plate, the catheter hub can be quickly detached and lifted out of the patient, then replaced with another hub. This process requires only a small incision and does not necessitate removal of the line assembly or the anchoring back plate, and can thus be accomplished through a small incision during a brief procedure.

After a hemodialysis treatment, the bicameral catheter hub and connected line assembly are flushed with saline solution. Additional saline may be injected into the conduit tunnels of the catheter hub to promote continued clearing of the lumens. The present invention agitates intralumenal fluid via acoustic wave propagation, thereby reducing fluid stagnation. Fluid flow outside the lumens is also encouraged by the propagation of acoustic waves throughout the surrounding environment. Improved blood flow generally result in reduced bacterial adhesion to tissue and blood cells, and consequently results in reduced risk of infection within the affected vasculature. Further, blood stream stagnation increases a patient's risk of thrombus formation. The present hemodialysis catheter device reduces this risk by generating low-energy acoustic waves, which improve extralumenal fluid flow and create an environment inhospitable to thrombus formation. It can thus be understood that the catheter device is useful not only in the dissolution of vascular occlusions, but also in reducing the likelihood of occlusion formation.

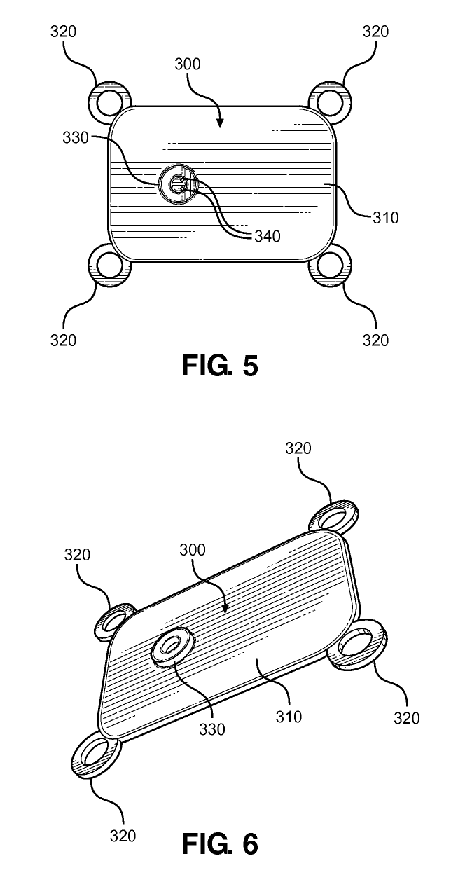

Referring now to FIG. 5, the top of an exemplary anchoring back plate 300 is shown. This portion of the catheter device secures to surrounding fascia, and serves as a pivot point for the catheter hub. The main body 310 of the back plate may be of any geometric shape known to one of ordinary skill in the art. Area of the main body should be equal to or greater than that of the catheter hub bottom to prevent same from rubbing against tissue during repositioning. As shown in FIG. 6, the top and bottom of the main body are smooth to reduce the risk of tissue abrasion.

A plurality of suture wings 320 extends outward from the main body 310. During installation of the device, sutures are sewn around the wings and into the underlying fascia to firmly secure the back plate in position. Placement and numbering of suture wings with respect to the main body may vary according to the back plate shape. In the depicted example, the main body is rectangular to conform to the generally rectangular shape of the catheter hub and four suture wings are present at the corners of the main body. This illustration is for exemplary purposes only as any geometric design may be used n the construction of the anchoring back plate. In alternative embodiments, the suture wings may be replaced with apertures in the main body of the back plate.

An upstanding collar protrudes from the upper surface of the anchoring back plate main body 310. The upstanding collar 330 is the female portion of a mating pair that enables removable securement of the catheter hub to the back plate. The collar is disposed near, but preferably not on the lower edge of the anchoring back plate. This positioning encourages pivoting of the catheter hub about the collar with minimal tugging on the line assembly. Placement of the collar, and resultantly the catheter hub pivot point, near the center or top edge of the back plate could result in partial lumen displacement whenever the hub is repositioned. Further protection against lumen displacement is provided by vertical extrusions 340 within the interior of the upstanding collar. These extrusions extend inward from the inner wall of the collar, and are disposed at opposing forty-five degree angles from the longitudinal axis of the main body 310. A male extension on the catheter hub body has a vertical extrusion that rests between the vertical extrusions of the upstanding collar when the catheter hub is in place. These extrusions form stops that prevent the catheter hub from moving past a ninety-degree range of motion. Other ranges of degree may be incorporated into the device, but the catheter hub should not be permitted to pivot at over 180 degrees as high angles of rotation will result in severe tugging at the lumens, and possible discomfort or injury to the patient.

In some embodiments a spring biased catch is disposed within the upstanding collar, such that insertion of the male extension with downward force results in securement of the extension within the collar. Similarly, collapsible flanges may be employed, along with any other form of quick insertion attachment means known in the art. The catheter hub is also easily removable and should be disengaged via the exertion of downward force on the spring-biased catch. In this way, a physician can quickly press the hub down into place and then remove it at a later time by pressing downward and lifting up on the hub body. Magnetic attachment mechanisms must be avoided as they are incompatible with magnetic resonance imaging (MRI) devices and may make it difficult for patients to undergo medical imaging. In an alternative embodiment, the male extension may snap into permanent connection with the upstanding collar, necessitating removal of the back plate in order to remove the catheter hub. All versions of the securement mechanism must permit single axis rotation about the point of connection.

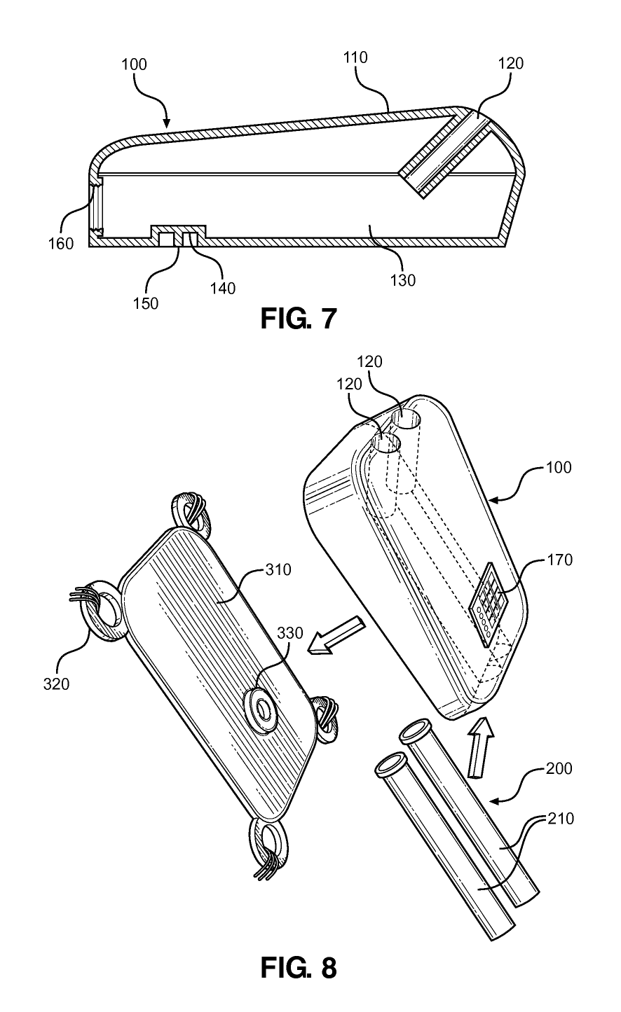

The male extension is illustrated in the catheter hub cross-section of FIG. 7. An exemplary catheter hub 100 is shown having a hub body 110 that houses conduit tunnels 130 extending between injection ports 120 and connection ports 160. The injection ports extend inward from an upper end of the hub body, which may protrude slightly above the rest of the hub body. These ports extend inward at an angle of approximately thirty degrees from the horizontal and facilitate fluid communication between the conduit tunnels and an inserted needle. At the lower, opposing end of the hub body are connection ports that enable removable securement of the line assembly to the hub body. Screw threading may be used to connect the lumens of the line assembly to the hub body, as well as any other connection means known in the art to one of ordinary skill.

Along the underside of the catheter hub body 110 is a recess 140 with a peg-like protrusion. This protrusion is the male extension 150, which forms a mating pair with the upstanding collar of the anchoring back plate. As discussed above, the male extension is received by and retained within the upstanding collar while the catheter device is in use. The male extension may have a conical, cylindrical, or irregular shape so long as it has a cylindrical cross-section that permits rotation about a vertical axis. Like the upstanding ring, the male extension is positioned near but not at the lower edge of the catheter hub. Placement of the recess and associated male extension can vary according to the intended orientation of the hub with respect to the anchoring back plate. By way of example, the recess and male extension may be disposed near a side edge of the hub body if the designated orientation of the catheter hub is orthogonal to the anchoring back plate.

Once the catheter device is properly installed and associated wounds healed, vascular access may be achieved as needed. During hemodialysis sessions, needles will be inserted through the skin, into the injection ports to facilitate blood withdrawal and introduction. Delivery of therapeutic agents such as antibiotics and pain medications may also take advantage of the catheter hub's access points. The present invention's pivotable hub-to-back plate connection allows repositioning of the injection ports underneath a patient's skin. Light pressure on a patient's skin along a lateral edge of the catheter hub will result in angular translation of the hub body and attached injection ports. Such translation may be accomplished by a physician or the patient themselves, making the present catheter device suitable for in-home dialysis treatments. Regular repositioning of the injection ports will reduce the frequency with which a particular region of skin must receive injections. Injection sites may be permitted to heal fully before being used again, thereby reducing a patient's discomfort during each hemodialysis session.

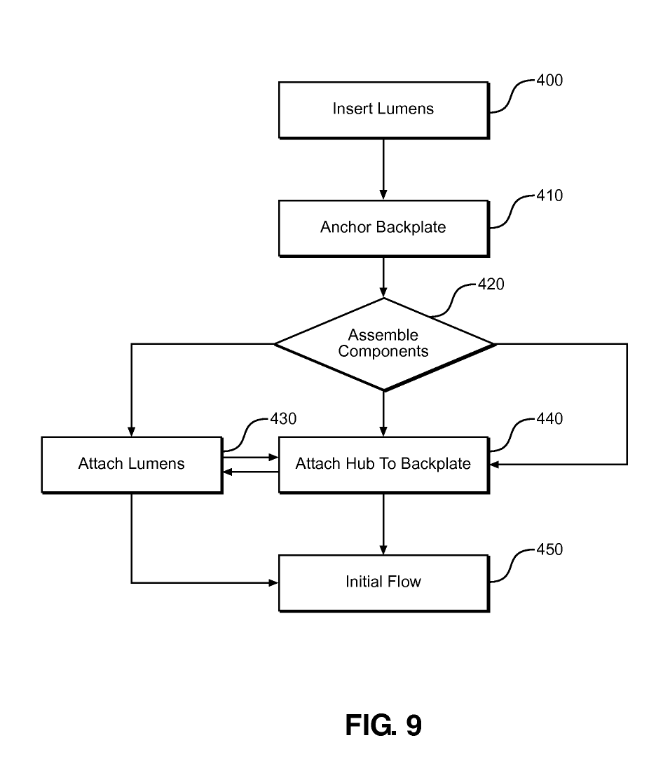

Surgical installation of the catheter device is demonstrated in FIG. 8 and the associated method depicted in the flow chart of FIG. 9. Central venous catheters are generally implanted in a patient's chest during a surgical procedure. While placement may vary according to the assessment of the operating physician, the procedure described herein is directed towards implantation within a vein of the patient's chest. Variations on the placement of the catheter with respect to a patient's anatomy will be apparent to one of ordinary skill in the art and thus the use of the present invention is not limited to central venous implantation.

The first step in implanting the catheter device is to insert the line assembly 400 into a selected vein. This may be accomplished using a guide wire that is fed into the target pathway, and then feeding the lumens 210 down over the guide wire. The second end of the lumens should be inserted first, leaving the first ends of the lumens proximal to the intended positioning of the catheter hub 100 free. The first end portion of the line assembly 200 is left exposed to aid in connection with the connection ports of the catheter hub.

At step 410 the anchoring back plate is secured in position. The main body 310 of the anchoring back plate 300 is positioned over a target securement region such that the upstanding collar 330 is directed upwards and positioned proximal to the first end of the lumens 210. Upon achievement of desired positioning, an operating physician sutures the back plate to underlying fascia at the suture wings 320. The back plate should be sufficiently secured such that the suture wings are not easily lifted away from the underlying tissue.

Next, the catheter components are assembled 420. Order of the following two steps is interchangeable and will depend on patient anatomy and the operating physician's preference. In these steps, lumens are attached to the catheter hub 430 and the hub is secured to the anchoring back plate 440.

Attachment of the line assembly 430 includes connecting the first ends of the lumens to catheter hub connection ports. By way of example, the first ends may be individually screwed into the connection ports, pressed into the ports until the securement means engages or the like. This places line assembly lumens in electrical communication with the control circuitry and power source 170 disposed on the catheter hub. Consequently, the acoustic wave generation by the transducer arrays disposed within the lumens may begin. The connection between lumens and catheter hub also places the lumens in fluid communication with the hub and its injection ports, rendering the lines ready for flushing.

The catheter hub 100 is removably secured to the installed anchoring back plate 300 via the mating pair of the upstanding collar 330 and the male extension. Prior to attachment, the catheter hub is oriented with the injection ports directed upward and distal from the lower edge if the anchoring back plate. Likewise, the connection ports should be positioned proximal to the exposed first ends of the lumens 210. The hub body is then gently lowered down onto the anchoring back plate until the male extension is fully inserted into the upstanding collar, which may include engagement of a catch or other removable securement means. If the hub is properly attached to the back plate, it can be arcuately pivoted forty-five degrees in either lateral direction.

Lastly, the assembled catheter device is flushed 450 to ensure proper fluid movement through the hub and lines. Needles are inserted into the injection ports 120 and a saline solution or sterilizing agent is introduced into the catheter hub. If the device is properly installed, the saline solution will travel through the conduit tunnels and connection ports, into the lumens. Transducer arrays formed of piezoelectric crystal elements generate low-energy acoustic waves throughout the catheter lumens and surrounding vascular environment. Wave propagation encourages fluid flow within the lumen and thereby ads in flushing the catheter lines. Once the device is properly flushed with saline solution or a sterilizing agent, the wound in the skin may be closed and the procedure ended.

The implanted hemodialysis catheter is used to facilitate regular filtration of the blood of patients suffering from varying degrees of renal failure. On a periodic basis, the patient undergoes a hemodialysis treatment in which both of the injection ports are utilized to achieve vascular access and establish blood removal and delivery lines. One lumen is established as the removal line and the remaining lumen is established as the delivery line. Blood is removed through the injection port and sent to a filtration machine, which removes impurities and passes the blood to the injection port associated with the delivery line. Treatment continues until a predetermined volume of blood is successfully filtered. During treatments, needles are inserted into the injection ports and thus acoustic wave generation is terminated. Transducer array agitation will continue upon removal of the needles.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An implantable hemodialysis catheter device comprising:
a catheter hub having a hub body that houses a pair of injection ports and a pair of conduit tunnels, wherein said hub body has a pair of connection ports disposed at a lower end, and wherein said connection ports are placed in fluid communication with said injection ports via said conduit tunnels;
an anchoring back plate having a plurality of suture points and a point of pivotable, removable, connection to said catheter hub;
a line assembly, comprising a pair of lumens, each of said lumens having a first end and a second end, wherein said first ends of said lumens are removably secured to the connection ports of said catheter hub body.

2) The implantable hemodialysis catheter device of claim 1, wherein the device further comprises:
an acoustic wave generator comprising, a transducer array integrally disposed within each of said lumens of said line assembly, wherein said transducer array is electrically coupled to a power source and an activator disposed upon said catheter hub.

3) The implantable hemodialysis catheter device of claim 2, wherein said acoustic wave generator is configured to produce vibrations within and surrounding said line assembly lumens.

4) The implantable hemodialysis catheter device of claim 2, wherein each of said transducer arrays is a plurality of piezoelectric crystals electrically connected via one or more wires.

5) The implantable hemodialysis catheter device of claim 4, wherein at least a portion of said piezoelectric crystals are configured to resonate within a frequency range of 100 to 300 kHz.

6) The implantable hemodialysis catheter device of claim 4, wherein at least a portion of said piezoelectric crystals are configured to resonate within a range of 300 to 700 kHz.

7) The implantable hemodialysis catheter device of claim 4 wherein a first portion of said piezoelectric crystals are configured to resonate within a frequency range of 100 to 300 kHz, and a second portion of said piezoelectric crystals are configured to resonate within a range of 300 to 700 kHz

8) The implantable hemodialysis catheter device of claim 2, wherein insertion of a needle into one or both of said injection ports terminates operation of said acoustic wave generator, and removal of said needle initiates operation of said acoustic wave generator.

9) The implantable hemodialysis catheter device of claim 2, wherein operation of said acoustic wave generator is initiated and terminated via a depressible button disposed on said catheter hub, wherein said button is electrically coupled to said power source.

10) The implantable hemodialysis catheter device of claim 1, wherein said injection ports are extend inward from the exterior of said hub body at an angle of approximately thirty degrees from the horizontal.

11) The implantable hemodialysis catheter device of claim 1, wherein said point of pivotable, removable connection permits rotation about a vertical axis.

12) The implantable hemodialysis catheter device of claim 1, wherein said point of pivotable removable connection is disposed near a lower edge of said anchoring back plate.

13) The implantable hemodialysis catheter device of claim 1 wherein said point of pivotable, removable connection comprises a male extension protruding downward from a recess disposed on an underside of said catheter hub, and an upstanding collar disposed along an upper surface of said anchoring back plate, wherein said upstanding collar is adapted to receive and removable engage said male extension.

14) The implantable hemodialysis catheter device of claim wherein said catheter hub is prevented from rotating past a predetermined angle of rotation.

15) The implantable hemodialysis catheter device of claim 14, wherein said predetermined angle of rotation is a ninety degree angle centered on the longitudinal axis of said anchoring back plate.

16) A method of surgically implanting a hemodialysis catheter, comprising the steps of:
running a second end of a line assembly comprised of at least two lumens into a target vein and leaving a first end of said line assembly exposed;
surgically securing an anchoring back plate to underlying tissue such that a point of connection adapted to pivotably and removably engage with a catheter hub, is directed upwards and positioned proximal to said first end of said line assembly;
assembling catheter components;
flushing said hemodialysis catheter with a fluid solution after assembly.

17) The method of surgically implanting a hemodialysis catheter of claim 16, wherein assembling catheter components comprises:
connecting said line assembly first end to connection ports disposed on said catheter hub;
removably securing said catheter hub to said anchoring back plate at said point of pivotable, removable connection such said catheter hub is arcuately repositionable about said point of connection.

18) The method of surgically implanting a hemodialysis catheter of claim 16, wherein assembling catheter components comprises:
removably securing said catheter hub to said anchoring back plate at said point of pivotable, removable connection such said catheter hub is arcuately repositionable about said point of connection
connecting said line assembly first end to connection ports disposed on said catheter hub.

19) The method of surgically implanting a hemodialysis catheter of claim 16, further comprising:
activating an acoustic wave generator integrated into said catheter device, prior to flushing said hemodialysis catheter.

20) An implantable hemodialysis catheter device, comprising:
a catheter hub;
an anchoring back plate having a plurality of suture points, wherein said catheter hub is removably and pivotably secured to said anchoring back plate;
a line assembly comprising a pair of μ, wherein said line assembly is removably secured to said catheter hub;
an acoustic wave generator comprising, a transducer array integrally disposed within each of said lumens of said line assembly, wherein said transducer array is electrically coupled to a power source and an activator disposed upon said catheter hub.
